(19) United States
(12) Patent Application Publication
(10) Pub. No.: US 2025/0072915 A1
(43) Pub. Date: Mar. 6, 2025

(54) APPARATUS FOR PREPARING A MEDULLARY CAVITY OF A BONE FOR AN IMPLANT, AN INSTRUMENT COMPRISING THE APPARATUS

(71) Applicant: Aesculap AG, Tuttlingen (DE)

(72) Inventors: Martin Nonnenmann, Wurmlingen (DE); Sebastian Weindel, Emmingen-Liptingen (DE)

(21) Appl. No.: 18/810,652

(22) Filed: Aug. 21, 2024

(30) Foreign Application Priority Data
Aug. 28, 2023 (DE) 10 2023 123 069.7

(51) Int. Cl. A61B 17/17

(52) U.S. Cl. CPC A61B 17/1735; A61B 17/1764

(57) ABSTRACT

An instrument and apparatus for preparing a medullary cavity of a bone for an implant includes a drill for producing a bore in the medullary cavity, a fastening device for a rasp for producing an opening in the medullary cavity, and a housing. The drill and the fastening device are each translationally movable relative to the housing. The apparatus also includes a guide configured to guide the drill in a translational direction of movement of the drill for producing the bore and the rasp in the translational direction of movement or in substantially the translational direction of movement for producing the opening.

### CROSS-REFERENCE TO RELATED APPLICATION(S)

This application claims priority under 35 U.S.C. § 119 to German Application No. 10 2023 123 069.7, filed on Aug. 28, 2024, the content of which is incorporated by reference herein in its entirety.

### FIELD

The present disclosure relates to an apparatus for preparing a medullary cavity of a bone for an implant and an instrument comprising the apparatus.

### BACKGROUND

To prepare a medullary cavity of a bone, there are known drills for preparing a bore for receiving a rotationally symmetrical part of an implant and rasps for preparing openings arranged laterally of the bore for anchoring the implant in a medullary cavity of the bone arranged laterally of the rotationally symmetrical part.

### SUMMARY

The device according to the present disclosure allows for preparing the bore and the openings arranged laterally thereto with an instrument.

The apparatus for preparing a medullary cavity of a bone for an implant comprises a drill for producing a bore in the medullary cavity, a fastening device for a rasp for producing an opening in the medullary cavity and a housing, wherein the drill is arranged so as to be movable relative to the housing in a translational manner, wherein the fastening device is arranged so as to be translationally movable relative to the housing, wherein the device comprises a guide, wherein the guide is designed to guide the drill in a translational direction of movement of the drill to produce the bore and the rasp in the translational direction of movement or lead substantially to the translational direction of movement to produce the opening.

The drill and the rasp are preferably arranged to be translationally movable to one another in the translational direction of movement or in substantially the translational direction of movement.

The guide preferably comprises a guide device arranged at least partially in the housing, which is designed to allow a movement of the drill in the translational direction of movement guided by the guide device.

The guide device is preferably arranged in a translationally movable manner with respect to the housing in the translational direction of movement or in substantially the translational direction of movement.

The apparatus preferably comprises a fastening device for the rasp, wherein the rasp is attached to the fastening device.

The rasp is preferably releasably attached to the fastening device.

The guide device preferably comprises the fastening device.

The guide device is preferably configured to absorb impact energy for moving the guide device in the translational direction of movement and to transmit it to the fastening device for moving the fastening device in the translational direction of movement or in substantially the translational direction of movement.

The guide device is preferably at least partially arranged in the housing, wherein the housing has a projection that is designed to function as a stop for the guide device.

The fastening device is preferably at least partially arranged in the housing, wherein the housing has a projection that is designed to function as a stop for the guide device.

The guide device preferably comprises an impact device for absorbing the impact energy.

The housing preferably comprises a recess for the impact device, in which the impact device is arranged so that it is movable in the translational direction of movement with respect to the housing up to an end of the recess designed as a stop.

The impact device preferably comprises a surface that protrudes from the housing at least in sections radially to the translational direction of movement or to the surface essentially projecting from the translational direction of movement for receiving the impact energy, wherein the impact device has an undercut on the rear side of the surface.

The apparatus preferably comprises a coupling device, which is configured to couple the drill and the driving device during a movement opposite to the translational direction of movement.

The guide preferably comprises at least one part of a wall of the housing, which is designed to allow a movement of the rasp or the fastening device guided by at least one part of the wall in the translational direction of movement or in substantially the translational direction of movement.

The apparatus preferably comprises a stop acting in the direction or opposite of the direction of the translational movement of the drill relative to the housing.

The drill preferably comprises a shank, wherein at least one part of the shank is arranged in the guide device, wherein the part of the shank has a tapered shape, wherein the guide device has a constriction that forms the stop.

The housing preferably comprises on its outside an area which is designed as a handle.

The housing preferably comprises a raised portion on the outside, which is arranged between the area designed as a handle and an area of the apparatus in which a head of the drill or the rasp is arranged.

Preferably, at least one notch is arranged in the area designed as a handle.

The rasp preferably comprises at least two blades, wherein a recess for the drill is arranged between the two blades.

The housing preferably comprises a deflection device for absorbing impact energy against the translational direction of movement or against essentially the translational direction of movement, wherein the deflection device comprises a surface projecting from the housing at least in sections radially to the translational direction of movement or to essentially the translational direction of movement for absorbing the impact energy.

An instrument for preparing a medullary cavity of a bone for an implant comprises at least two interchangeable drills or at least two interchangeable rasps.

The instrument preferably comprises a preparation plateau.

### BRIEF DESCRIPTION OF THE DRAWINGS

Further advantageous embodiments can be found in the following descriptions and the drawings. The following is shown in the drawings:

FIG. 1 shows a schematic, perspective illustration of parts of an exemplary embodiment of an apparatus for preparing a medullary cavity of a bone for an implant;

FIG. 2 shows an exploded view of parts of the exemplary embodiment of the apparatus,

FIG. 3 shows a schematic, perspective view of an exemplary sash rasp,

FIG. 4 shows a schematic, perspective view of an exemplary preparation plateau,

FIG. 5 shows a schematic, perspective view of an exemplary adapter of a motor system,

FIG. 6 shows a side view of the apparatus,

FIG. 7 shows a frontal view of the apparatus,

FIG. 8 shows a first section through the apparatus,

FIG. 9 shows a second section through the apparatus,

FIGS. 10a to 10d each show a schematic illustration of a use of the apparatus based on the example of a tibia preparation.

### DETAILED DESCRIPTION

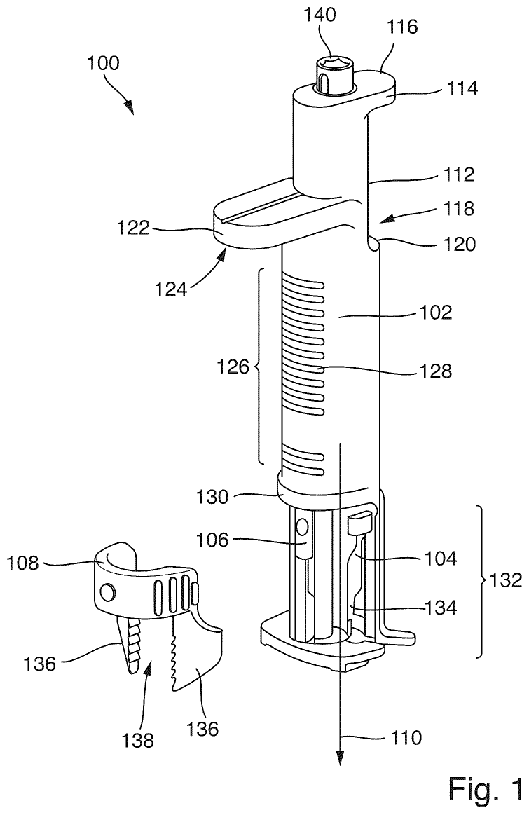

FIG. 1 shows a schematic, perspective illustration of parts of an apparatus 100 for preparing a medullary cavity of a bone for an implant. The apparatus 100 is a medical instrument.

The apparatus 100 for preparing the medullary cavity is described using the example of an implant for a knee, i.e., based on the example of a knee endoprosthesis. The apparatus 100 is embodied, for example, for preparing the medullary cavity of a tibia.

The apparatus 100 comprises a housing 102, a drill 104 for producing a bore in the medullary cavity and a fastening device 106 for a rasp 108 for producing an opening in the medullary cavity.

The drill 104 is arranged to be translationally movable relative to the housing 102. The fastening device 106 is arranged to be translationally movable relative to the housing 102.

The drill 104 and the rasp 108 are arranged to be translationally movable relative to one another in the translational movement direction 110 or in substantially the translational movement direction.

The apparatus 100 comprises a guide. The guide is designed to guide the drill 104 in the translational movement direction 110 of the drill 104 to produce the bore.

The guide is designed to guide the rasp 108 in the translational movement direction or in substantially the translational movement direction to produce the opening.

In the example, the guide comprises at least one part of a wall of the housing 102, which is designed to allow a movement of the rasp 108 or the fastening device 106 in the translational movement direction 110 or in substantially the translational movement direction, guided by at least a part of the wall.

The guide comprises, for example, a guide device 112 arranged at least partially in the housing 102. The guide device 112 is designed to allow a movement of the drill 104 in the translational movement direction 110 guided by the guide device 112. The guide device 112 is designed to allow a movement of the drill 104 against the translational movement direction 110 guided by the guide device 112. The guide device 112 is designed to guide the drill 104 in its drilling axis in or against the translational direction of movement 110. The guide device 112 is designed to allow a rotational movement of the drill 104 guided by the guide device 112 about its drilling axis.

In the example, the guide device 112 is at least partially arranged in the housing 102.

The guide device 112 is preferably arranged in a translationally movable manner with respect to the housing 102 in the translational direction of movement 110 or in substantially the translational direction of movement.

The rasp 108 is attached to the fastening device 106. In the example, the rasp 108 is releasably attached to the fastening device 106. Rasps 108 are for example provided in different sizes which can optionally be detachably fastened to the fastening device 106. It can be provided that the apparatus 100 is designed with a rasp 108 that is permanently attached to the fastening device 106.

The guide device 112 is designed to absorb impact energy for moving the guide device 112 in the translational movement direction 110 and to transfer it to the fastening device 106 for moving the fastening device 112 in the translational movement direction 110 or in substantially the translational movement direction.

In an exemplary embodiment of the apparatus 100, the guide device 112 comprises the fastening device 106.

In an exemplary embodiment of the apparatus 100, the fastening device 106 is designed as a tappet.

In the exemplary embodiment, the fastening device 106 is at least partially arranged in the housing 102.

The guide device 112 comprises an impact device 114 for absorbing impact energy.

In the exemplary embodiment, the impact device 114 comprises a surface 116 which protrudes from the housing 102 at least in sections radially to the translational direction of movement 110 or substantially to the translational direction of movement for absorbing the impact energy.

In the exemplary embodiment, the housing 102 comprises a recess 118 for the impact device 114, in which the impact device 114 is arranged in a manner so that it is movable in translational direction of movement 110 relative to the housing 102. The impact device 114 is arranged to be movable, for example, up to an end 120 of the recess designed as a stop.

It can be provided that the impact device 114 has an undercut on the rear side of the surface 116, in particular an undercut that can be easily gripped with one hand. The undercut allows the guide device to be easily retracted.

In the exemplary embodiment, the housing 102 comprises a deflection device 122 for absorbing impact energy counter to the translational direction of movement 110 or essentially counter to the translational direction of movement.

The deflection device 122 comprises, for example, a surface 124 which protrudes radially from the housing 102 at least in sections radially to the translational direction of movement 110 or substantially to the translational direction of movement for absorbing the impact energy.

In the exemplary embodiment, the housing 102 comprises on its outside an area 126 which is designed as a handle. For example, at least one notch 128 is arranged in the area 126 which designed as a handle.

The housing 102 comprises a raised portion 130 on the outside, which is arranged between the area 126 designed as a handle and an area 132 of the apparatus 100 in which a head 134 of the drill 104 or the rasp 108 is arranged.

This at least one notch 128 in the handle improves the grip even with wet, greasy or gloves otherwise contaminated with bodily fluids.

This raised portion 130 separates the area 126 of the housing 102, which is not suitable as a grip area due to movable parts.

In the exemplary embodiment, the rasp 108 comprises at least two blades 136. There may be only one blade provided or more than two blades, e.g. three blades or four blades.

A recess 138 for the drill 104 is arranged between the blades 136.

The drill 108 comprises a shaft 140. At least a portion of the shank 140 is arranged in the guide device 112.

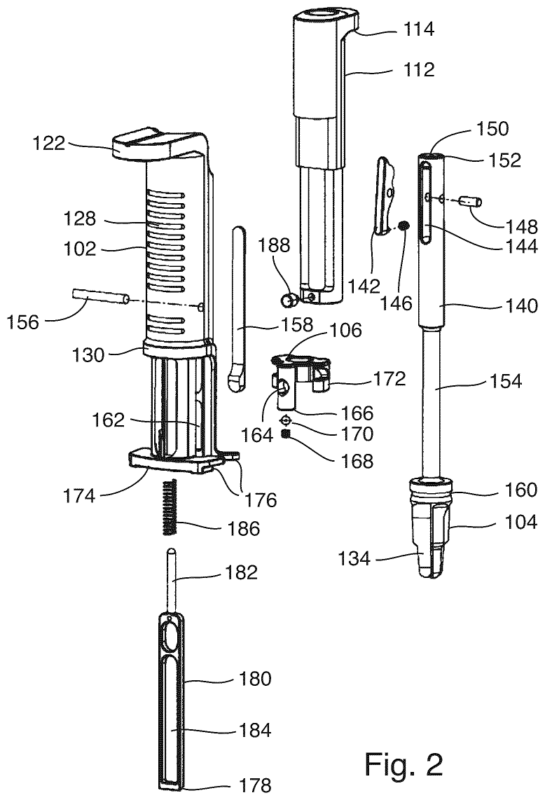

FIG. 2 shows an exploded view of parts of the exemplary embodiment of the apparatus 100.

The apparatus 100 comprises e.g. a coupling device, which is configured to couple the drill 104 and the impact device 114 during a movement opposite to the translational direction of movement 110.

In the exemplary embodiment, the coupling device comprises a latch 142, which is arranged in a recess 144 in the shaft 140 of the drill 104.

The drill 104 is connected to the impact device 114 via the latch 142. The drill 104 and the impact device 114 are connected by the latch 142 in such a way that the drill 104 and the driving device 114 can be returned together into an initial position after preparation of the bone. The latch 142 is designed, for example, as a rocker spring-loaded by a spring 146. In the exemplary embodiment, the rocker comprises a rocker bar, which is formed by a pin 148. In the exemplary embodiment, the pin 148 is held in opposite bores in the side walls of the recess 144. In the exemplary embodiment, the spring 146 is a pressure spring that is supported on a rear wall of the recess 144.

In the exemplary embodiment, the shaft 140 comprises an adapter 150 for a motor system. In the exemplary embodiment, the adapter 150 is arranged on a front side of the drill 104. The adapter 150 comprises an opening, for example, a hexagon socket, in the shaft 140. In the exemplary embodiment, the opening extends from the front side to the recess 144.

In the area of the adapter 150, the shaft 140 comprises a hole between the pin 148 and the front side, which connects the opening and the recess 114. In the exemplary embodiment, one end of the latch 142 is positioned so it can be moved outward through the opening when the motor system is inserted into the adapter 150. This causes the latch 142 to open automatically when the motor system is inserted into the opening in the shaft 140 of the drill 104.

Alternatively, the drill 104 can have an interface that allows direct coupling to a motor system without an adapter.

It can be provided that the motor system is also an element of the apparatus 100.

It can be provided that the apparatus 100 comprises a stop acting in or opposite of the translational movement 110 for the translational movement of the drill 104 relative to the housing 102. For example, the part of the shaft 140 which is arranged in the guide device 112, has a taper 154. For example, the guide device 112 has a pin 156 which is held in the region of the taper 154 by two bores in the housing 102 and runs transversely to the direction of movement 110, which forms the two stops for the non-tapered parts of the shaft 140.

In the exemplary embodiment, the head 134 of the drill 104 comprises a cutting geometry, which is designed in such a way that a bore can be prepared for a rotationally symmetrical part of the implant in the tibia.

In the exemplary embodiment, the drill 104 is held in a starting position in the housing 102 by a spring 158 integrated into the housing 102. The spring 158 is, for example, designed as a leaf spring. In the exemplary embodiment, the spring 158 engages in a groove 160, which is arranged on the periphery of the drill 104. The groove 160 is arranged, for example, between the head 134 and the tapering 154. The spring 158 and the groove 160 hold the drill 104 firm in the starting position by means of a form-locking connection up to a defined force. The form-locking connection is designed in such a way that it unlocks automatically once a defined force is acting on the drill 104 in the drill direction.

It can be provided that the housing 102 has at least one opening 162 arranged in the area 132 of the apparatus 100 in which a head 134 of the drill 104 or the rasp 108 is arranged. The weight of the housing 102 is reduced and its cleaning possibility is improved by the at least one opening 162.

In the exemplary embodiment, the fastening device 106 comprises a first bore 164 extending transversely to the translational direction of movement 110 for receiving a pin of the rasp 108. In the exemplary embodiment, a second bore 166 extending in the translational direction of movement 110 is arranged in the first bore 164. A spring 168 and a ball 170, which is pressed by the spring 168 into the first bore 164 are arranged in the second bore 166. The ball 170 and the spring 168 are designed so that the ball 170 snaps into a groove on the pin of the blade rasp when the pin of the blade rasp is inserted into the first bore 164.

The fastening device 106 comprises a bracket 172 arranged transversely to the translational direction of movement 110, which is designed to engage around the drill 104. In the exemplary embodiment, the bracket 172 comprises two ends, onto which a groove arranged transversely to the translational direction of movement 110 can be pushed onto the rasp 108.

In the exemplary embodiment, the housing 102 comprises a first part of an interface 174 to a preparation plateau on its side opposite to the adapter 150 in the translational direction of movement 110. The first part of the interface 174 has an opening for the head 134 of the drill 104 and a plate surrounding the opening with at least one projection 176 in an edge region of the plate.

The apparatus 100 comprises a particular beveled snap nose 178. The snap nose 178 is arranged on a snap latch 180 which is arranged in the housing 102a in a movable manner in the translational direction of movement 110. The snap lock 180 comprises, on its side opposite to the snap nose 178 in the translational direction of movement 110, a first pin 182 extending in the translational direction of movement 110. Between the pin 182 and the snap nose 178, the snap latch 180 comprises an oblong hole 184 extending in the translational direction of movement 110. The first pin 184 is designed to guide a spiral spring 186 that is pushed onto the first pin 184. In the exemplary embodiment, the housing 102 comprises a sleeve extending in the translational direction of movement 110, into which the pin 184 can be immersed. The spiral spring 186 is supported on a stop arranged in the housing 102 and it presses the latch 180 out of the housing 102 until it reaches a stop. The latch 180 is coupled to the guide device 112 via a second pin 188, which extends the guide device 112 transversely to the translational direction of movement 110.

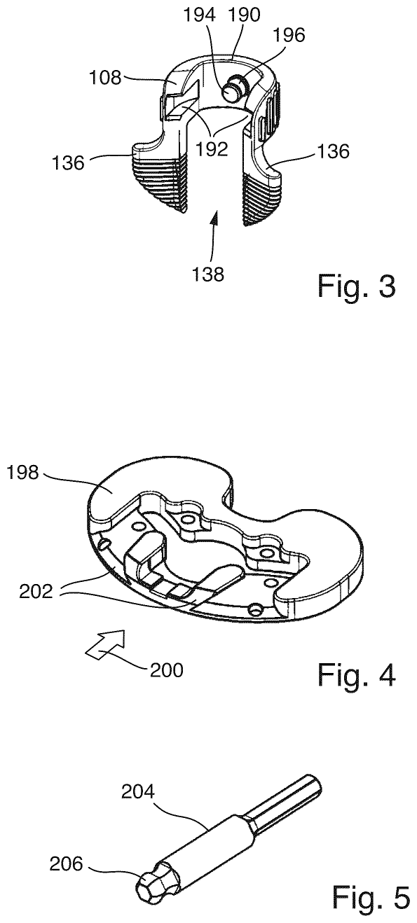

FIG. 3 shows a schematic, perspective illustration of an exemplary blade rasp 108.

The blade rasp 108 comprises a bracket 190, which is designed to encompass the fastening device 106. In the exemplary embodiment, the bracket 190 comprises one connecting groove 192 on each side in the bracket 190, which is designed to be pushed onto the bracket 176 of the fastening device 106, in particular onto its ends.

The two brackets are designed to transfer the impact energy to the blade rasp 108.

The blade rasp 108 comprises a pin 194, which is configured to engage in the first bore 164 of the fastening device 106. The pin 194 comprises a groove 196 circumferential radially around the pin 194, which is designed to receive the ball 170. The ball 170 snaps into the groove 196 on the pin 194 of the blade rasp 108 when the pin 194 is inserted into the first bore 164.

The brackets 176, 190, the pins 194, and the groove 192 are connecting elements that run perpendicular to the direction of impact. The snapping the blade rasp 108 into the place with these connecting elements is sufficient as a locking mechanism between the blade rasp 108 and the apparatus 100 and, after the use of the apparatus 100, also enables a simple removal of the blade rasp 108 without having to actuate separate control elements. It can be provided that all or only some of the connecting elements are provided.

FIG. 4 shows a schematic, perspective view of an exemplary preparation plateau 198. The preparation plateau 198 is, for example, designed for the preparation of a tibia.

In the exemplary embodiment, the apparatus 100 and the preparation plateau 198 are configured to adapt the apparatus 100 from the anterior direction 200 to the preparation plateau 198.

In contrast to adaptation from the proximal direction, adaptation from the anterior direction leads to fewer conflicts with the femur and soft tissues.

The preparation plateau 198 comprises a second part of the interface 202.

In the exemplary embodiment, the interface is designed as an angular dovetail guide.

Due to the two angles, the interface has a lot of play at the beginning of the coupling movement and can therefore also be joined between the two components even if there is a slight offset or angular error. When connecting, the apparatus 100 automatically centers itself on the preparation plateau 198.

The apparatus 100 and the preparation plateau 198 can alternatively also be designed with an interface that can be coupled and decoupled in the proximal direction.

Alternatively, the apparatus 100 may comprise a support surface, which is designed in such a way that it can be placed directly on the bone without a preparation plateau 198. Rough structures or spikes integrated into the support surface can optionally prevent possible slipping. Optional bores may be provided in the apparatus 100, which are designed to enable fixation of the apparatus 100 directly on the bone by means of screw pins or impact pins.

If the apparatus 100 is coupled as far as it will go, it will lock automatically via the in particularly beveled snap nose 178.

FIG. 5 shows a schematic, perspective view of an exemplary adapter 204 of the motor system.

The adapter 204 of the motor system comprises, for example, an external hexagonal adapter 206. In the exemplary embodiment, a hexagonal end 206 of the adapter 204 is designed spherical in shape. This allows the adapter 204 to transmit rotational movements even if the angles of the axes of the drill and the motor system do not exactly match.

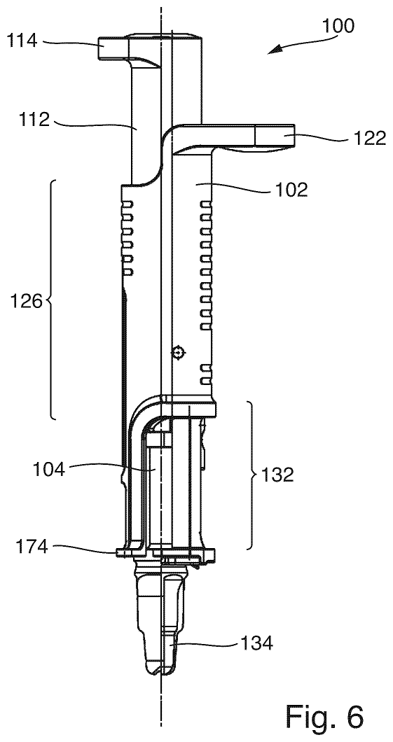

FIG. 6 shows a side view of the apparatus 100. In the side view, the head 134 of the drill 104 is pushed out of the housing 102 through the interface 174. This represents a working end position of the drill 104.

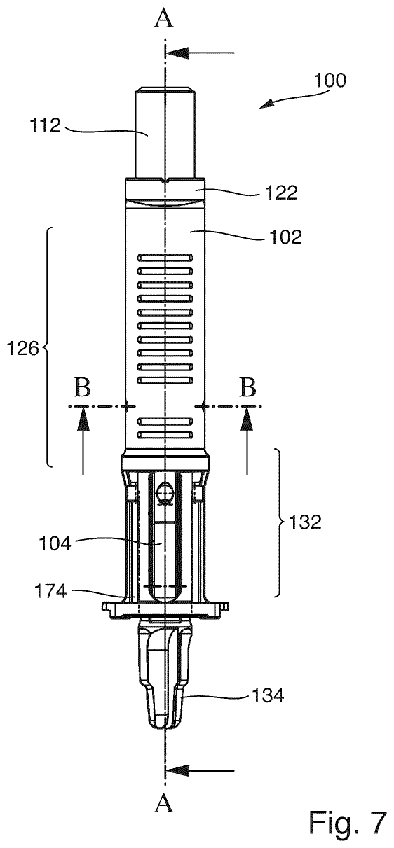

FIG. 7 shows a frontal view of the apparatus 100. In the frontal view, the drill 104 is in the working end position.

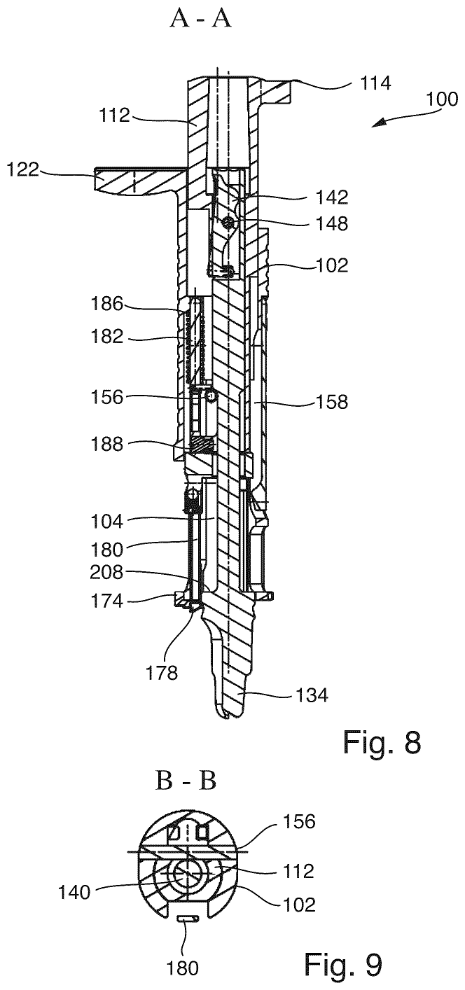

FIG. 8 shows a first section A-A through the apparatus 100, in which the drill 104 is arranged in the working end position.

The housing 102 has a projection 208.

The projection 208 Is configured as a depth stop for the guide device 112 or for the fastening device 106.

Since, in the respective working end position of the drill 104 and the guide device 112, the latch 142 of the drill engages in the guide device 112, the drill 104 is simultaneously retracted into the starting position by retracting the guide device 112. At the same time, the pin 188 at the guide device 112 pulls back the snap latch 180 and thus unlocks the preparation plateau 198 from the apparatus 100.

FIG. 9 shows a second section B-B through the apparatus 100 in the region of the pin 156, which forms the two stops for the non-tapered parts of the shaft 140 of the drill 104.

The stops are designed in such a way that the preparation depth suitable for the respective implant is ensured.

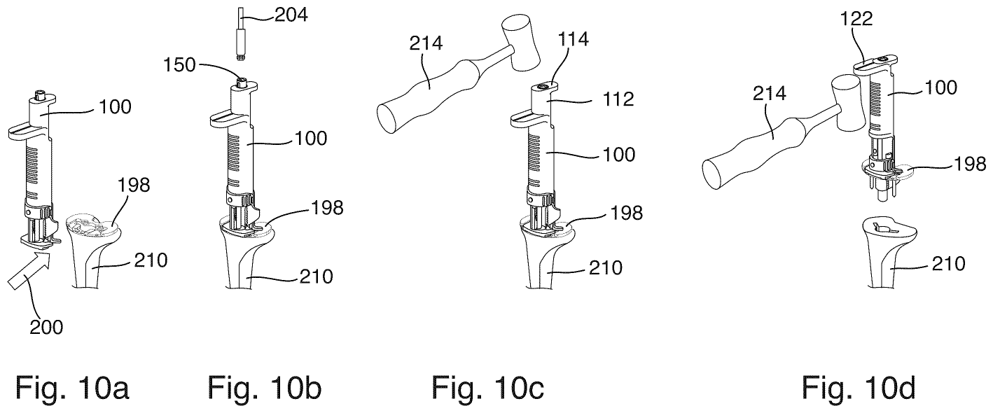

FIGS. 10a to 10d schematically show a use of the apparatus 100 using the example of a preparation of a tibia 210.

FIG. 10a shows the tibia 210 on which the preparation plateau 198 is already arranged. The apparatus 100 is adapted from the anterior direction 200 to the preparation plateau 198.

FIG. 10b shows the apparatus 100 arranged on the preparation plateau 198. In the exemplary embodiment, the snap nose 178 of the snap lock 180 is engaged in the preparation plateau 198. The drill hole in the tibia 210 is prepared using the drill 104. The motor system rotates the drill 104 via the adapter 204 of the motor system and the adapter 150 of the apparatus 100.

FIG. 10c shows the rasp 108 being driven in with a hammer 214. The impact plate 114 at the upper end of the apparatus 100 transmits the impact energy of the hammer 214 to the fastening device 106 for the rasp 108 via the guide device 112. As a result, the rasp 108 connected to the fastening device 106 is hammered into the tibia 210.

Since the drill 104 is also centrally positioned in the tibia 210 during the impaction process of the rasp 108, an angular stability is achieved during impaction that exceeds the stability of previously used separate drill and rasp instruments.

FIG. 10d shows the impact. The impact plate 122, which is permanently connected to the housing 102 of the apparatus 100, enables the complete apparatus 100, including drill 104 and rasp 108 to be impacted.

The apparatus 100 is also engaged in the preparation plateau 198 by means of the snap lock 180. Therefore, the drill 104, the rasp 108, and the preparation plateau 198 connected to the apparatus 100 can be removed in a work step.

The preparation plateau 198 may comprise anchoring pins for anchoring the preparation plateau 198 in the tibia 210. In this case, the anchoring pins are removed together with the preparation plateau 198.

It can be provided that the projections 176 are arranged on the housing 102 in such a way that they cover the anchoring pins in the preparation plateau 198. When the apparatus 100 swings out together with the preparation plateau 198, the anchoring pins cannot accidentally fall out.

The apparatus 100 or rather the apparatus 100 and the preparation plateau 198 can thus be transferred from the surgeon to the assistant as a unit together with the drill 104, the rasp 108, the preparation plateau 198 and, if applicable, the anchoring pins.

For an implant system that requires bores of different depths, drills 104 of different lengths can be provided, wherein one of the drills 104 is arranged in the housing 102 in a removable manner. Alternatively, an adjustable depth stop can be arranged in the housing for a non-detachable drill 104. Drills 104 with the same rotationally symmetrical geometry or with different geometries can be provided.

This allows a geometry for a rotationally symmetrical pin to be prepared for anchoring in the bone with drills of different lengths.

It can be provided that the apparatus 100 comprises one or more interfaces for a hammer having an impact weight. This means that a hammer with a guided impact weight can be used for driving in and/or removing.

For an implant system that requires openings of different depth to be prepared with the rasp 108, rasps 108 of different depths or an adjustable depth stop for the rasp 108 can be provided.

For an implant system that requires different arrangements of the blades of the rasp 108 that are positioned differently to one another or to the central bore, the blades 136 of the rasp 108 can be designed to be removable or non-removable on the apparatus 100 and/or adjustable in their position on the apparatus 100.

An instrument for preparing the medullary cavity comprises, for example, the apparatus 100, wherein at least two interchangeable rasps 108 of different length, width or different arrangement of the blades 136 are provided.

It can be provided that the apparatus 100 is designed for exchangeable drills, wherein the instrument comprises at least two exchangeable drills 104 of different lengths or with different diameters.

It can be provided that the instrument comprises the preparation plateau 198.

1. An apparatus for preparing a medullary cavity of a bone for an implant, the apparatus comprising:
a drill for producing a bore in the medullary cavity;
a fastening device for a rasp for producing an opening in the medullary cavity;
a housing; and
a guide,
the drill and the fastening device each being translationally movable relative to the housing,
the guide configured to guide the drill in a translational direction of movement for producing the bore, and
the guide configured to guide the rasp in the translational direction of movement or in substantially the translational direction of movement for producing the opening.

2. The apparatus according to claim 1, wherein the drill and the rasp are translationally movable relative to one another in the translational direction of movement or in substantially the translational direction of movement.

3. The apparatus according to claim 1, wherein the guide comprises a guide device that is at least partially arranged in the housing and designed to allow a movement of the drill in the translational direction of movement guided by the guide device.

4. The apparatus according to claim 3, wherein the guide device is translationally movable relative to the housing in the translational direction of movement or in a manner that is substantially movable in the translational direction of movement.

5. The apparatus according to claim 3, further comprising a fastening device for the rasp, wherein the rasp is attached to the fastening device.

6. The apparatus according to claim 5, wherein the rasp is releasably attached to the fastening device.

7. The apparatus according to claim 5, wherein the guide device comprises the fastening device.

8. The apparatus according to claim 5, wherein the guide device is designed to absorb impact energy for movement of the guide device in the translational direction of movement and to transmit the guide device to the fastening device for movement of the fastening device in the translational direction of movement or in substantially the translational direction of movement.

9. The apparatus according to claim 8, wherein the guide device is arranged at least partially in the housing, and wherein the housing has a projection that is configured as a stop for the guide device.

10. The apparatus according to claim 8, wherein the fastening device is at least partially arranged in the housing, and wherein the housing has a projection that is designed as a stop for the fastening device.

11. The apparatus according to claim 10, wherein the drill comprises a shaft, wherein at least one part of the shaft is arranged in the guide device, wherein the at least one part of the shaft comprises a taper, and wherein the guide device comprises a constriction that forms the stop.

12. The apparatus according to claim 8, wherein the guide device comprises an impact device for absorbing the impact energy.

13. The apparatus according to claim 12, wherein the housing comprises a recess for the impact device, in which the impact device is arranged in a manner allowing movement with respect to the housing in the translational direction of movement up to an end of the recess designed as a stop.

14. The apparatus according to claim 12, wherein the impact device comprises a surface which projects from the housing at least in sections radially to the translational direction of movement or to substantially the translational direction of movement for absorbing the impact energy, wherein the impact device comprises an undercut on a rear side of the surface.

15. The apparatus according to claim 12, wherein the apparatus comprises a coupling device designed to couple the drill and the impact device during a movement counter to the translational direction of movement.

16. The apparatus according to claim 1, wherein the guide comprises at least a part of a wall of the housing, which is designed to allow a movement of the rasp or the fastening device guided by at least a part of the wall in the translational direction of movement or in substantially the translational direction of movement.

17. The apparatus according to claim 1, wherein the apparatus comprises a stop acting in or counter to the translational direction of movement for translational movement of the drill relative to the housing.

18. The apparatus according to claim 1, wherein the housing comprises an outside and an area on the outside that forms a handle.

19. The apparatus according to claim 18, wherein the housing comprises a raised portion on the outside, the raised portion arranged between said area on the outside and a region of the apparatus, in which a head of the drill or the rasp is arranged.

20. The apparatus according to claim 19, wherein at least one notch is arranged in the region, the at least one notch being configured as the handle.

21. The apparatus according to claim 1, wherein the rasp comprises at least two blades, wherein a recess for the drill is arranged between the at least two blades.

22. The apparatus according to claim 1, wherein the housing comprises a deflection device for absorbing an impact energy counter to the translational direction of movement or counter to the translational direction of movement, wherein the deflection device has a surface protruding from the housing at least in sections radially to the translational direction of movement or to substantially the translational direction of movement for absorbing the impact energy.

23. An instrument for preparing a medullary cavity of a bone for an implant, the instrument comprising:
the apparatus according to claim 1; and
at least two exchangeable drills or at least two exchangeable rasps.

24. The instrument according to claim 23, further comprising a preparation plateau.
